(19) United States
(12) Patent Application Publication
(10) Pub. No.: US 2003/0065386 A1
(43) Pub. Date: Apr. 3, 2003

(54) Radially expandable endoprosthesis device with two-stage deployment

(76) Inventor: Kevin Shaun Weadock, Princeton, NJ (US)

Correspondence Address: Scully, Scott, Murphy & Presser, 400 Garden City Plaza, Garden City, NY, 11530, US

(21) Appl. No.: 09/968,481

(22) Filed: Sep. 28, 2001

(51) Int. Cl. A61F 2/24; A61F 2/06

(52) U.S. Cl. 623/2.38; 623/1.15; 623/23.75

(57) ABSTRACT

A radially expandable endoprosthesis device and method of deployment with an at least two stage deployment capability. More particularly, the device pertains to an annularly expandable heart valve prosthesis which is adapted for the long-term treatment of valvular diseases in infants, children and adolescents. The device is constituted of a combination of superelastic alloys and bioresorabable materials which facilitates the devices to undergo multistage deployments at predetermined intervals while emplaced in the body vessels or lumens of patients.

### BACKGROUND OF THE INVENTION

1. Field of the Invention

The present invention relates to a radially expandable endoprosthesis device with an at least two stage deployment capability and, more particularly, pertains to an annularly expandable heart valve prosthesis which is adapted for the long-term treatment of valvular diseases in infants, children and adolescents.

Basically, radially expandable endoprosthesis devices are employed in connection with the insertion and positioning of stents or stent-grafts into corporeal vessels, such as arteries or the like, and generally are constituted of stainless steel or nitinol (nickel-titanium alloy) or similar alloys. In the instance in which an endoprothesis employed as a stent, it is adapted to counteract acute vessel spasms which are frequently encountered in the emplacement of nitinol (nickel-titanium alloy) stents in arteries or body vessels. In coronary arteries, any secondary enlargement of the stent would be adapted to serve for offsetting contractile forces which may result from intimal hyperplasia; however, the prior art pursuant to the state of the technology, does not address itself to this aspect. When employed in connection with abdominal aortic aneurysms (AAA), current stent-graft devices merely concern themselves with anchoring devices the stent-graft in its location of emplacement.

Heretofore, in the prior art, the problems encountered the use of such endoprosthesis devices have been addressed by various methods and physical and biological means. Thus, in intimal hyperplasia of coronary arteries, additional angioplasty, or in the use of chemicals and pharmaceutical preparates, such as various drugs or radio-isotopes, these may be readily employed in order to attempt to reduce the hyperplasia. Furthermore, the emplacement of external bands around abdominal aortic aneurysms (AAA) which are treated with stent-grafts has also been employed in order to account for any aneurysmal progression which may occur at a site which has been thought to be free of disease. When employed in pediatric heart valve disease cases, secondary surgeries are frequently needed in order to replace the smaller-sized valve prosthesis as the infant or child grows, as a result of an increase in the heart valve sizes requiring larger-sized prosthesis, this being at times the cause of severe discomfort, and even morbidity and increased morbidity rates for such tender patients.

2. Discussion of the Prior Art

As disclosed in Duerig et al. U.S. Pat. No. 6,179,878, a composite self-expanding stent device incorporates a restraining element, in which a restraint sleeve is generally formed of a shape memory alloy, such as binary nickel titanium alloy, referred to generally as nitinol, and wherein restraint can be provided in the form of either sleeve, covering a mesh or perforated sheet. In that instance, the restraining element can be formed of a polymeric material which, in any event is not considered to be possessed of a property to enable the stent device to undergo multiple dimensionally changing configurations at predetermined intervals in time so as provided a device with an at least two-stage deployment in a patient.

Lenker et al. U.S. Pat. No. 6,176,875 discloses an endoluminal prosthesis and methods in the use thereof, which provides for limited radial expansion in controlled mode. However, the stent-graft construction illustrated and described therein is primarily equipped with a belt which may frangible or expansible in order to allow for further or subsequent expansion of the implanted or emplaced stent-graft device. This device also fails to provide for a combination of super-elastic shape memory alloys such as nitinol, and bioresorbable medical materials which enable the devices to undergo at least a two-stage or multiple deplacement stages at predetermined intervals in time.

Finally, Lock et al. U.S. Pat. No. 5,383,926 discloses an expandable endoprosthesis device which is constituted of the combination of a memory alloy, possibly such as nitinol, with an expansion limiting structure which is selectively removable in order two subsequently allow for further radial expansion of the emplaced device, whereby the expansion limiting structure can be constituted of a dissolvable or severable band-like material. Although this endoprosthesis device may generally incorporate bioresorbable materials, the device described in this patent is not adapted for heart valve prostheses, particularly such as are intended for pediatric applications, which will enable the treatment of valvular diseases in children, whereby the annulus of the heart valve prosthesis can be caused over periods of time to expand as the child grows, thereby obviating the need for further surgical procedures normally required in order to substitute larger-sized heart valve prosthesis structures or devices in the growing patients.

### SUMMARY OF THE INVENTION

Accordingly, in order to provide an endoprosthesis device which is adapted to essentially provide for a multi-stage deployment and which facilitates a radially and annular expansion which may be required during continual use thereof, the inventive device, such as a stent, stent-graft, or pursuant to a preferred embodiment, a heart valve prosthesis particularly for pediatric case is drawn to a novel combination of superelastic or shape memory alloys and bioresorbable materials, which enables the devices to undergo multiple or at least two-stage configurations at predetermined time intervals depending upon the type of material employed in conformance with the needs of patients in which the devices are deployed. The bioresorbable materials may also serve as reservoirs for therapeutic agents, such as antibiotics, anticoagulants, and cytostatic drugs.

In one aspect, the device may comprise a coronary stent which is capable of having at least one deployment stage, and that is constituted of a superelastic material with a bioresorbable coating or constraint structure operatively combined therewith. This type of stent may be suitable for counteracting or addressing problems relative to initmal hyperplasmia when utilized in coronary vessels, and can also be employed for the stenting of other body vessels subjected to abdominal aortic aneurysms (AAA) when there is encountered the need to maintain contact with a dynamic vessel wall of a body vessel or lumen. In those last-mentioned instances, a stent for the counteracting the effects of the aneurysms, when constituted of the combination of superelastic alloys and bioresorabable materials can offset post-deployment aneurismal dilatation.

In a particularly preferred embodiment of the invention, the endoprosthesis device, which is constituted of a combination of a superelastic alloy and bioresorabable material, is in the configuration of a heart valve prosthesis especially adapted for pediatric medical uses, and which can be made to expand in at least two-steps of its deployment as the infant or child grows, over an extended period of time. In that connection, the endoprosthesis device may be constructed so as to incorporate various types of polymer systems in order to afford multiple stage deployments, wherein particular types of polymers may degrade at time intervals of, for example, ranging from about 6 months to about 200 months after the implanting of the device in the pediatric patient. In particular, such a system is useful in long-term heart valve prostheses, whereas contrastingly another system may utilize a polymer which absorbs in 15 minutes and which is useful in implanting anastomotic devices.

Accordingly, it is a primary object of the present invention to provide an endoprosthesis device which is constituted of a combination of superelastic alloys and bioresorbable materials which facilitates the devices to undergo multistage deployments at predetermined intervals while emplaced in the body vessels or lumens of patients.

Another object of the present invention is to provide an endoprosthesis device as described herein, wherein the device may undergo at least two-stage deployment so as to assume different or expanded annular or radial dimensions at predetermined time intervals responsive to degradation of bioresorbable components of the device which have been combined with a superelastic alloy.

A more specific object of the present invention is to provide an endoprosthesis device which is constituted of a heart valve prosthesis for pediatric medical applications, wherein the annulus of the valve prosthesis can be constructed so as to expand in at least two stages of deployment over periods of time during the growth of an infant or child, and wherein the device is constituted of a novel combination of superelastic alloy-materials and bioresorbable materials preferably selected from polymer systems.

### BRIEF DESCRIPTION OF THE ACCOMPANYING DRAWINGS

Reference may now be made to the following detailed description of embodiments of the invention, taken in conjunction with the accompanying drawings; in which:

FIGS. 1a-1d disclose, generally diagrammically, cross-sectional transverse views in the stages of deployment of a coronary stent constituted of a superelastic alloy combined with a bioresorabable restraining polymer which addresses itself to counteracting the effects of stenosis due to intimal hyperplasia;

FIGS. 2a-2d illustrate; diagrammatically in longitudinal sectional views, various stages as to the manner in which a stent comprised of a superelastic alloy and bioresorabable material can offset post-deployment residual aneurysmal dilation encountered which may be at the neck of a stent-graft used for abdominal aortic aneurysms (AAA); and

FIGS. 3a and 3b illustrate, respectively, the two-stage deployment offered by the construction of the endoprosthesis device as a heart valve possessing an expandable annular ring or neck portion, and which is especially adapted for use in long-term pediatric medical applications.

### DETAILED DESCRIPTION OF PREFERRED EMBODIMENTS OF THE INVENTION

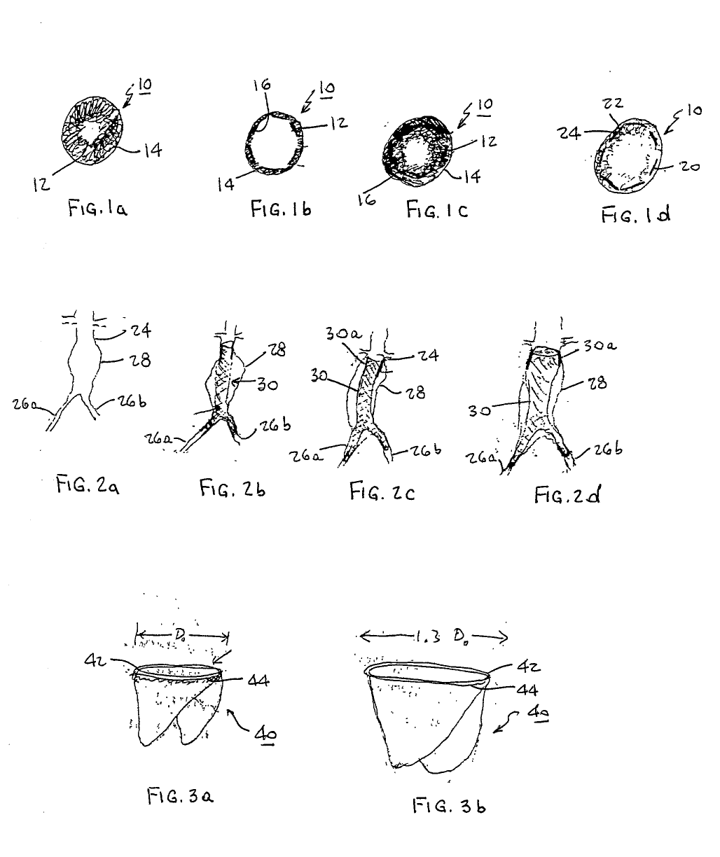

Reverting more specifically to FIGS. 1a through 1d of the drawings; FIG. 1a illustrates a transverse cross-sectional view through a coronary artery 10 in the pre-stenting stage; showing the interior buildup of plaque 12 along the artery wall 14.

FIG. 1b illustrates the artery 10 shown in a post-stenting stage wherein there is illustrated a stent 16 forming a wall interiorly of the plaque 12 and vessel or coronary artery wall 14; whereby as shown in FIG. 1c there may be encountered in-stent restenosis caused by intimal hyperplasia tending to occlude the artery.

In contrast with the foregoing, FIG. 1d illustrates a stent 20 pursuant to the inventive construction incorporates the combination of a suitable bioresorabable restraining polymer 22 with a superelastic alloy 24 on which it may be coated, such as nitinol (nickel-titanium alloy) or the like which may address the effects of intimal hyperplasia. In particular, the secondary radially expanded deployment of the stent 20 as a result of the gradual absorption or degradation of the bioresorbable restraining polymer 22 which allows the superelastic alloy the freedom to expand, provides for an effective lumen or blood flow increase; whereby the body vessel diameter itself may increase only slightly.

The bioresorbable restraining polymers which may be employed in this connection may be PLA-PGA copolymer systems, polytyrosine systems, or other suitable polymer systems which can be modified to afford different absorption rates and degrading stages. It is also possible to use two different bioresorbable polymer systems in combination with each other (and with the superelastic alloy) which afford further secondary and tertiary deployment stages to the implanted device.

Referring to FIGS. 2a through 2d of the drawings, in FIG. 2a there is illustrated a bifurcated blood vessel comprising aortic portion 24 extending between the heart and a pair of iliac branches 26a, 26b showing an abdominal aortic aneurysm 28 prior to stenting. As illustrated in FIG. 2b, a suitable abdominal aortic aneurysm (AAA) stent or bifurcated aorto-iliac vascular prosthesis 30 which is constituted of the combination of the superelastic alloy material and bioresorbable polymers system or systems, which may be in the form of a stent-graft construction possesses suitable anastomosis devices (not shown) adapted to exclude the aneurysm, is deployed in the body vessel or lumen.

As illustrated in FIG. 2c of the drawings, in the event that the stent-graft structure does not include the bioresorbable materials, the device fails to exclude the aneurysm as a result of encountered post-deployment dilatation of the proximal neck 30a of the device; whereas contrastingly by utilizing the combined materials, such as the superelastic alloy and bioresorbable polymers of the invention, as shown in FIG. 2d of the drawings, the resorption and degradation over time of the polymer material allows the stent-graft to enter a second stage of an additional expansion, thereby forming a protection against the aneurysm and any potential failure of the implanted stent-graft structure or device.

Reverting to the preferred embodiment of the invention, as illustrated in FIGS. 3a and 3b of the drawings, this diagrammatically discloses a heart valve prosthetic device 40 which is particularly adapted for pediatric applications with infants, children or adolescents who are still subject to growth in heart and heart valve dimensions over protracted periods of time.

As shown in FIG. 3a, the valve prosthesis 40 includes a ring construction or annulus 42 constituted in combination of a superelastic alloy, such as nitinol or the like, and a bioresorbable material 44 coated thereon which is adjusted for the growth of a pediatric patient. As implemented, the system of the material 44 utilizes a bioresorbable restraining polymer in combination with the superelastic alloy material 42, such as a PLA-PGA copolymer system, polytyrosine system, or other suitable polymer system or combinations thereof, which can be suitably modified for different absorption rates, such as by degrading, for example, at time intervals ranging from between about 6 months to 200 months, so as to allow for the second-stage in expansion of the prosthesis. As indicated, combinations of two different polymer systems can be employed to afford secondary and tertiary deployment stages at specified time intervals.

Thus, as shown in FIG. 3a of the drawings, the annulus of the device as initially implanted in a child, for example of 2 years in age, may possesses a ring or neck diameter D₀ constituted of a prosthesis of a nitinol ring 42 coated with the polymer system 44.

The secondary expansion, as shown in FIG. 3b, which is permitted by the present system, shows the heart valve prosthesis with a diameter of at least 1.1 D₀ expanded as a result of the polymer absorption, thereby enabling the valve device to be deployed in the body vessel or heart valve of the pediatric patient for extended periods of time during the growth of the patient, without necessitating further surgery for removal of the initial smaller device and substitution of a larger-sized heart valve device. This clearly lowers the risk of possible morbidity or complications due to any second surgical procedure which have been required for the installation of a larger valve pursuant to the current state in the medical technology.

From the foregoing, it becomes clearly apparent that the invention, wherein in particular a pediatric heart valve prosthesis is constituted of the combination of superelastic alloy, such as nitinol or the like, and bioresorbable materials comprising various polymers or polymer systems, counteracts deleterious or natural phenomena which may otherwise compromise the performance and efficacy of a two-stage deployable endoprosthetic device which is merely constituted of a superelastic alloy material without resorbable biological materials forming restraining elements degradable over specified periods of time.

While the invention has been particularly shown and described with respect to preferred embodiments thereof, it will be understood by those skilled in the art that the foregoing and other changes in form and details may be made therein without departing from the spirit and scope of the invention.

What is claimed is:

1. A radially expandable endoprosthesis having an at least two-stage deployment capability, said endoprosthesis comprising a heart valve prosthesis including a valve annulus which subsequent to deployment in a patient is expandable from a first diameter to at least a second larger diameter within a specified interval of time.

2. A radially expandable endoprosthesis as claimed in claim 1, wherein said endoprosthesis is constituted of a combination of a superelastic alloy and a bioresorbable material.

3. A radially expandable endoprosthesis as claimed in claim 2, wherein said superelastic alloy comprises nitinol.

4. A radially expandable endoprosthesis as claimed in claim 2, wherein said bioresorbable material comprises a coating on said superelastic alloy.

5. A radially expandable endoprosthesis as claimed in claim 2, wherein said bioresorbable material comprises a restraint means on said superelastic alloy.

6. A radially expandable endoprosthesis as claimed in claim 2, wherein said bioresorbable material is constituted of a polymer system possessing specified rates of resorption so as to enable said annulus to enter said at least second stage of additional radial expansion.

7. A radially expandable endoprosthesis as claimed in claim 2, wherein the specified interval of time for a resorption of the resorbable material is selected to be in the range of about 6 months to about 200 months at which said annulus expands to the at least second larger diameter.

8. A radially expandable endoprosthesic as claimed in claim 7, wherein said at least second larger diameter is at least 1.1 times the size of said first diameter.

9. A radially expandable endoprosthesis as claimed in claim 2, wherein said endoprosthesic comprises a coronary stent for the counteracting of restenosis.

10. A radially expandable endoprosthesis as claimed in claim 2, wherein said endoprothesis comprises a stent for the stenting of aortic aneurysms.

11. A radially expandable endoprosthesis as claimed in claim 2, wherein said bioresorbable material is selected to enable said annulus to undergo secondary and tertiary stages of expansion.

12. A radially expandable endoprosthesis as claimed in claim 6, wherein said polymer system is selected from the group of materials consisting of PLA-PGA copolymer systems, polytyrosine systems, and combinations of differing polymer systems for controllably varying the resorption rates thereof.

13. A radially expandable endoprosthesis as claimed in claim 6, wherein, wherein said polymer system contains a therapeutic agent.

14. A radially expandable endoprosthesis as claimed in claim 13, wherein said therapeutic agent selectively comprises an antibiotic, cytostatic or anticoagulant.

15. A method of deploying a radially expandable endoprosthesis having an at least two-stage deployment capability, said endoprosthesis comprising a heart valve prosthesis including a valve annulus which subsequent to deployment in a patient is expandable from a first diameter to at least a second larger diameter within a specified interval of time.

16. A method of deploying a radially expandable endoprosthesis as claimed in claim 15, wherein said endoprosthesis is constituted of a combination of a superelastic alloy and a bioresorbable material.

17. A method of deploying radially expandable endoprosthesis as claimed in claim 16, wherein said superelastic alloy comprises nitinol.

18. A method of deploying a radially expandable endoprosthesis as claimed in claim 16, wherein said bioresorbable material comprises a coating on said superelastic alloy.

19. A method of deploying a radially expandable endoprosthesis as claimed in claim 16, wherein said bioresorbable material comprises a restraint means on said superelastic alloy.

20. A method of deploying a radially expandable endoprosthesis as claimed in claim 16, wherein said bioresorbable material is constituted of a polymer system possessing specified rates of resorption so as to enable said annulus to enter said at least second stage of additional radial expansion.

21. A method of deploying a radially expandable endoprosthesis as claimed in claim 16, wherein the specified interval of time for a resorption of the resorbable material is selected to be in the range from about 6 months to about 200 months at which said annulus expands to the at least second larger diameter.

22. A method of deploying a radially expandable endoprosthesic as claimed in claim 21, wherein said at least second larger diameter is at least 1.1 times the size of said first diameter.

23. A method of deploying a radially expandable endoprosthesis as claimed in claim 16, wherein said endoprosthesic comprises a coronary stent for the counteracting of restenosis.

24. A method of deploying a radially expandable endoprosthesis as claimed in claim 16, wherein said endoprothesis comprises a stent for the stenting of aortic aneurysms.

25. A method of deploying a radially expandable endoprosthesis as claimed in claim 16, wherein said bioresorbable material is selected to enable said annulus to undergo secondary and tertiary stages of expansion.

26. A method of deploying a radially expandable endoprosthesis as claimed in claim 20, wherein said polymer system is selected from the group of materials consisting of PLA-PGA copolymer systems, polytyrosine systems, and combinations of differing polymer systems for controllably varying the resorption rates thereof.

27. A method of deploying a radially expandable endoprosthesis as claimed in claim 20, wherein said system contains a therapeutic agent.

28. A method of deploying a radially expandable endoprosthesis as claimed in claim 27, wherein said therapeutic agent selectively comprises an antibiotic, cytostatic or anticoagulant.
